(19) United States
(12) Patent Application Publication
(10) Pub. No.: US 2021/0277430 A1
(43) Pub. Date: Sep. 9, 2021

(54) FERMENTATION PROCESS FOR THE PRODUCTION OF LIPIDS

(71) Applicant: LanzaTech, Inc., Skokie, IL (US)

(72) Inventors: Sean Dennis Simpson, Skokie, IL (US); Christophe Daniel Mihalcea, Skokie, IL (US); Robert John Conrado, Skokie, IL (US)

(21) Appl. No.: 16/812,967

(22) Filed: Mar. 9, 2020

(51) Int. Cl. C12P 7/64; C12P 7/62

(52) U.S. Cl. CPC C12P 7/6427; C12P 7/62

(57) ABSTRACT

The disclosure provides methods and systems to produce lipid products from a gaseous substrate using a two-stage fermentation process. The method comprises providing a gaseous substrate comprising CO, CO₂ or H₂ or mixtures thereof, to a first bioreactor containing a culture or one or more microorganisms and fermenting the substrate to produce acetate. The acetate from the first bioreactor is then provided to a second bioreactor, where it is used as a substrate for fermentation to lipids by one or more microalgae. Tail gas from the second bioreactor is recycled to the first bioreactor.

### FIELD OF THE DISCLOSURE

This disclosure related to a process comprising a two-stage system to produce one or more lipid products from a gaseous feedstock.

### BACKGROUND

The global energy crisis has caused increased interest in alternative approaches to production of fuels. Biofuels for transportation are attractive replacements for gasoline and are rapidly penetrating fuel markets as low concentration blends. Biomass derived biofuel production has emerged as a major approach in increasing alternative energy production and reducing greenhouse gas emissions. The production of biofuels from biomass enables energy independence has been shown to enhance development of rural areas and enhance sustainable economic development.

First generation liquid biofuels utilise carbohydrate feedstocks such as starch, cane sugar, corn, rapeseed, soybean, palm and vegetable oils. First-generation feedstocks present a number of significant challenges. The cost of these carbohydrate feed stocks is influenced by their value as human food or animal feed, while the cultivation of starch or sucrose-producing crops for ethanol production is not economically sustainable in all geographies. The sustained use of these feedstocks as a source for biofuels would inevitably place great strain on arable land and water resources. Therefore, it is of interest to develop technologies to convert lower cost and/or more abundant carbon resources into fuels.

Second generation biofuels are those produced from cellulose and algae. Algae were selected to produce lipids due to their rapid growth rates and the ability of algae to consume carbon dioxide and produce oxygen.

One area that has seen increased activity is the microbial synthesis of lipids which comprise the raw materials required for bio fuel production. Numerous studies have demonstrated an ability to accumulate lipids through the use of oleaginous yeasts on different substrates such as industrial glycerol, acetic acid, sewage sludge, whey permeate, sugar cane molasses and rice straw hydrolysate. Again, these second generation biofuel technologies have encountered problems due to high production costs, and costs associated with the transport and storage of the feedstock.

It has been recognised that catalytic processes may be used to convert gases consisting of CO, CO₂, or hydrogen (H₂) into a variety of fuels and chemicals. Alternatively, microorganisms may be used to convert these gases into fuels and chemicals. The biological processes, although generally slower than thermochemical processes, have several advantages over catalytic processes, including higher specificity, higher yields, lower energy costs and greater resistance to poisoning.

The production of acetic acid, acetate and other products such as ethanol by the anaerobic fermentation of carbon monoxide, and/or hydrogen and carbon dioxide has been demonstrated. See, e.g., Balch et al, (1977) International Journal of Systemic Bacteriology, 27:355-361; Vega et al, (1989) Biotech. Bioeng., 34:785-793;Klasson et al (1990) Appl. Biochem. Biotech., 24/25: 1; among others.

Acetogenic bacteria, such as those from the genus Acetobacterium, Moorella, Clostridium, Ruminococcus, Acetobacterium, Eubacterium, Butyribacterium, Oxobacter, Methanosarcina, Methanosarcina, and Desulfotomaculum have been demonstrated to utilize substrates comprising H₂, CO₂ and/or CO and convert these gaseous substrates into acetic acid, ethanol and other fermentation products by the Wood-Ljungdahl pathway with acetyl co-A synthase being the key enzyme. For example, various strains of Clostridium ljungdahlii that produce acetate and ethanol from gases are described in WO 00/68407, EP 117309, U.S. Pat. Nos. 5,173,429, 5,593,886, and 6,368,819, WO 98/00558 and WO 02/08438. The bacterium Clostridium autoethanogenum sp is also known to produce acetate and ethanol from gases (Abrini et al, Archives of Microbiology 161, pp 345-351 (1994)).

Acetobacterium woodii, a strictly anaerobic, non-spore forming microorganism that grows well at temperatures of about 30° C., has been shown to produce acetate from H₂ and CO₂. Balch et al. first disclosed the bacterium A. woodii which grows by the anaerobic oxidation of hydrogen and reduction of carbon dioxide. Buschorn et al showed the production and utilisation of ethanol by A. woodii on glucose. Fermentation of A. woodii was performed at glucose/fructose concentrations of up to 20 mM. Buschorn et al found that when the glucose concentration was increased to 40 mM, almost half of the substrate remained when A woodii entered the stationary growth phase, and ethanol appeared as an additional fermentation product. Balch et al found that the only major product detected by the fermentation of H₂ and CO₂ by A. woodii was acetate according to the following stoichiometry; 4H₂+2CO₂->CH₃COOH+H₂O.

This disclosure provides a fermentation process and system that capitalizes on the production of acetate by incorporating a second bioreactor where acetate is the substrate to produce lipids. The disclosure further provides enhanced efficiency by recycling unconsumed CO₂ from the second bioreactor back to the first bioreactor while removing O₂ from the recycle.

### SUMMARY

One embodiment of the disclosure is directed to a method for producing at least one lipid product from CO₂ and H₂, the method comprising: receiving at least CO₂ and H₂ in a first bioreactor containing a culture of at least one first microorganism in a first liquid nutrient medium, and fermenting the gaseous substrate to produce acetate product in a first fermentation broth; passing at least a portion of the first fermentation broth to a second bioreactor containing a culture of at least one second microorganism in a second liquid nutrient medium, where the second microorganism is not the same as the first microorganism and is selected from Scenedesmus, Thraustochytrium, Japonochytrium, Aplanochytrium, Elina, and Labyrinthula, and fermenting the acetate product to produce at least one lipid product in a second fermentation broth; obtaining a tail gas comprising at least CO₂ and O₂ from the second bioreactor; and separating at least a portion of the O₂ from the tail gas and recycling at least a portion of the reminder of the tail gas to the first bioreactor.

The production rate of acetate in the first bioreactor may be at least 10 g/L/day. At least one of the first microorganisms in the first bioreactor may be Acetobacterium, Moorella, Clostridium, Pyrococcus, Eubacterium, Desulfobacterium, Carboxydothermus Acetogenium, Acetoanaerobium, Butyribacterium, Peptostreptococcus, Ruminococcus, Oxobacter, Methanosarcina, or any combination thereof. At least one of the first micoorganisms in the first bioreactor may be Acetobacterium woodii. At least one of the second microorganisms in the second bioreactor may be Thraustochytrium. The method may further comprise producing, from the at least one lipid product, at least one tertiary product selected from hydrogenation-derived renewable diesel (HDRD), fatty acid methyl esters (FAME), fatty acid ethyl esters (FAEE), and biodiesel. The method method may further comprise limiting at least one nutrient in the second liquid nutrient medium in the second bioreactor to increase lipid production. The limited nutrient may be nitrogen. At least one lipid product may be a polyunsaturated fatty acid. The polyunsaturated fatty acid may be an omega-3 fatty acid. The omega-3 fatty acid may be one or more of alpha-linolenic acid (ALA), eicosapentaenoic acid (EPA), and docosahexaenoic acid (DHA). The separating of at least a portion of the O₂ from the tail gas may be accomplished using pressure swing adsorption or scrubbing with a basic solution. The method may further comprise recycling the O₂ from the separation of at least a portion of the O₂ from the tail gas to the second bioreactor. The method may further comprise recovering a gaseous stream comprising CO₂ and H₂ from the first bioreactor and recycling the gaseous stream to the first bioreactor. The method may further comprise recycling at least a portion of the second fermentation broth to the first bioreactor. The method may further comprise removing the first microorganism from the first fermentation broth before passing at least a portion of the first fermentation broth to the second bioreactor and recycling the first microorganism to the first bioreactor. The method may further comprise removing the second microorganism from the second fermentation broth and recycling the second microorganism to the second bioreactor. The method may further comprise passing, to the first bioreactor, the remainder of the second fermentation broth after removing the second microorganism. The method may further comprise generating the H₂ using a water electrolyzer. The method may further comprise generating O₂ using a water electrolyzer and introducing the electrolyzer generated O₂ to the second bioreactor.

In one embodiment the gaseous substrate is a waste or off gas from an industrial process. In one embodiment the waste gas is selected from the group comprising tail gas from a hydrogen plant, coke oven gas, associated petroleum gas, natural gas, catalytic reformer gas, naphtha cracker offgas, refinery fuel gas, methanol plant tail gases, ammonia plant tail gases, and lime kiln gases.

The disclosure may also includes the parts, elements and features referred to or indicated in the specification of the application, individually or collectively, in any or all combinations of two or more of said parts, elements or features, and where specific integers are mentioned herein which have known equivalents in the art to which the disclosure relates, such known equivalents are deemed to be incorporated herein as if individually set forth.

### BRIEF DESCRIPTION OF THE DRAWINGS

FIG. 1 is a diagram of one embodiment of the disclosure having a two-fermenter system for production of lipids from CO and optionally H₂, or CO₂ and H₂, wherein a substantially oxygen-free CO₂ stream is recycled from the secondary bioreactor to the primary bioreactor.

FIG. 2 is a diagram of one embodiment of the disclosure having a two-fermenter system for production of lipids from CO and optionally H₂, or CO₂ and H₂, wherein a substantially oxygen-free CO₂ stream is recycled from the secondary bioreactor to the primary bioreactor and where the H₂ used in the primary bioreactor and the O₂ used in the secondary bioreactor are generated using an electrolyzer.

FIG. 3 is a graph showing the acetate concentration in a fermentation utilizing CO.

### DETAILED DESCRIPTION

The present disclosure generally relates to a process of producing lipids by a first fermentation of gaseous substrates containing CO, and/or CO₂ and H₂ to produce acetic acid/acetate followed by a secondary fermentation wherein the acetate is converted into lipids. “Acid”, as used herein, includes both carboxylic acids and the associated carboxylate anion, such as the mixture of free acetic acid and acetate present in a fermentation broth as described herein. The ratio of molecular acid to carboxylate in the fermentation broth is dependent upon the pH of the system. The term “acetate” includes both acetate salt alone and a mixture of molecular or free acetic acid and acetate salt, such as the mixture of acetate salt and free acetic acid present in a fermentation broth as may be described herein. The ratio of molecular acetic acid to acetate in the fermentation broth is dependent upon the pH of the system. “Lipid”, as used herein, includes fatty acids, glycolipids, sphingolipids, saccharolipids, polyketides, sterol lipids and prenol lipids. In a one embodiment, the lipid may be a polyunsaturated fatty acid, such as an omega-3 fatty acid (also called ω-3 fatty acid or n-3 fatty acid). The omega-3 fatty acid may be one or more of alpha-linolenic acid (ALA), eicosapentaenoic acid (EPA), and docosahexaenoic acid (DHA).

Permeate—substantially soluble constituents of the broth that pass through the separator and are not retained by the separator. The permeate will typically contain soluble fermentation products, by-products and nutrient solution.

Dilution rate—the rate of replacement of the broth in a bioreactor. The dilution rate is measured in the number of bioreactor volumes of broth that are replaced by nutrient medium per day.

The substrate of the first fermentation refers to a carbon and/or energy source for the microorganisms of the disclosure. For at least one microorganism, the substrate may be gaseous and comprise a C1-carbon source, such as CO, CO₂, and/or CH₄. In one embodiment the substrate comprises a C1-carbon source of CO or CO+CO₂. The substrate may further comprise other non-carbon components, such as H₂, N₂, or electrons.

In specific embodiments the substrate may comprise at least some amount of CO, such as about 1, 2, 5, 10, 20, 30, 40, 50, 60, 70, 80, 90, or 100 mol % CO. In other embodiments, the substrate may comprise a range of CO, such as about 20-80, 30-70, or 40-60 mol % CO. The substrate may comprise about 40-70 mol % CO (e.g., steel mill or blast furnace gas), about 20-30 mol% CO (e.g., basic oxygen furnace gas), or about 15-45 mol % CO (e.g., syngas). In some embodiments, the substrate may comprise a relatively low amount of CO, such as about 1-10 or 1-20 mol % CO. The microorganism of the disclosure typically converts at least a portion of the CO in the substrate to a product. In some embodiments, the substrate comprises no or substantially no (<1 mol %) CO.

In some embodiments the substrate may comprise some amount of H₂. For example, the substrate may comprise about 1, 2, 5, 10, 15, 20, or 30 mol % H₂. In particular embodiments, the presence of hydrogen results in an improved overall efficiency of the fermentation process. In some embodiments, the substrate may comprise a relatively high amount of H₂, such as about 60, 70, 80, or 90 mol % H₂. In further embodiments, the substrate comprises no or substantially no (<1 mol % ) H₂.

In some embodiments, the substrate may comprise some amount of CO₂. For example, the substrate may comprise about 1-80 or 1-30 mol % CO₂. In some embodiments, the substrate may comprise less than about 20, 15, 10, or 5 mol % CO₂. In another embodiment, the substrate comprises no or substantially no (<1 mol %) CO₂. Often, when the substrate comprises CO₂, the substrate also comprises H₂.

Although the substrate is typically gaseous, the substrate may also be provided in alternative forms. For example, the substrate may be dissolved in a liquid saturated with a CO-containing, CO2-containing and/or H2-containing gas using a microbubble dispersion generator. By way of further example, the substrate may be adsorbed onto a solid support.

The substrate and/or C1-carbon source may be a waste gas obtained as a byproduct of an industrial process or from some other source, such as from automobile exhaust fumes or biomass gasification. In certain embodiments, the industrial process is selected from the group consisting of ferrous metal products manufacturing, such as a steel mill manufacturing, non-ferrous products manufacturing, petroleum refining, coal gasification, electric power production, carbon black production, ammonia production, methanol production, and coke manufacturing. In these embodiments, the substrate and/or C1-carbon source may be captured from the industrial process before it is emitted into the atmosphere, using any convenient method.

The substrate and/or C1-carbon source may be syngas, such as syngas obtained by gasification of coal or refinery residues, gasification of biomass or lignocellulosic material, or reforming of natural gas. In another embodiment, the syngas may be obtained from the gasification of municipal solid waste or industrial solid waste.

The composition of the substrate may have a significant impact on the efficiency and/or cost of the reaction. For example, the presence of oxygen (O₂) may reduce the efficiency of an anaerobic fermentation process. Depending on the composition of the substrate, it may be desirable to treat, scrub, or filter the substrate to remove any undesired impurities, such as toxins, undesired components, or dust particles, and/or increase the concentration of desirable components.

In some embodiments, the substrate may be syngas, and the syngas composition may be improved to provide a desired or optimum H₂:CO:CO₂ ratio. The syngas composition may be improved by adjusting the feedstock being fed to the gasification process. The desired H₂:CO:CO₂ ratio is dependent on the desired fermentation product of the fermentation process. By way of example, if the desired product is ethanol, the optimum H₂:CO:CO₂ ratio would be:

$${( x )}_{1} {( y )}_{1} ( \frac{x - 2 y}{\text{?}} ) , \\ \text{?} \text{indicates text missing or illegible when filed}$$

where [symbol] in order to satisfy the stoichiometry for ethanol production

$$( x ) {H}_{2} + ( y ) CO + ( \frac{x - 2 y}{\text{?}} ) {CO}_{2} \to ( \frac{x + y}{\text{?}} ) C \text{?} H \text{?} OH + ( \frac{x - y}{\text{?}} ) {H}_{2} O . \\ \text{?} \text{indicates text missing or illegible when filed}$$

Operating the fermentation process in the presence of hydrogen has the added benefit of reducing the amount of CO₂ produced by the fermentation process. For example, a gaseous substrate comprising minimal H₂ will typically produce ethanol and CO₂ by the following stoichiometry [6CO+3H₂O→C₂H₅OH+4CO₂]. As the amount of hydrogen utilized by the C1-fixing bacterium increases, the amount of CO₂ produced decreases [e.g., 2CO+4H₂→C₂H₅OH+H₂O].

When CO is the sole carbon and energy source for ethanol production, a portion of the carbon is lost to CO₂ as follows:

6CO+3H₂O→C₂H₅OH+4CO₂ (ΔG°=−224.90 kJ/mol ethanol)

As the amount of H₂ available in the substrate increases, the amount of CO₂ produced decreases. At a stoichiometric ratio of 2:1 (H₂:CO), CO₂ production is completely avoided.

5CO+1H₂+2H₂O→1C₂H₅OH+3CO₂ (ΔG°=−204.80 kJ/mol ethanol)

4CO+2H₂+1H₂O→1C₂H₅OH+2CO₂ (ΔG°=−184.70 kJ/mol ethanol)

3CO+3H₂→1C₂H₅OH+1CO₂ (ΔG°=−164.60 kJ/mol ethanol)

Broth bleed—the portion of the fermentation broth removed from a bioreactor that is not passed to a separator.

Separator—a module that is adapted to receive fermentation broth from a bioreactor and pass the broth through a filter to yield a retentate and a permeate. The filter may be a membrane, e.g. cross-flow membrane or a hollow fibre membrane.

In a first stage of the process, the gaseous substrate comprising CO₂ and H₂ or comprising CO and optionally H₂, is anaerobically fermented to produce at least one acid. In the second stage of the process, the at least one acid from the first stage is introduced to a second bioreactor containing a culture of at least a second microorganism. The second microorganism may be at least one microalgae. The at least one acid is aerobically converted by the second microorganism to produce one or more lipid products. Fermenting, fermentation process or fermentation reaction and similar terms as used herein, are intended to encompass both the growth phase and product biosynthesis phase of the process. As is described further herein, in some embodiments the bioreactor may comprise a first growth reactor and a second fermentation reactor. As such, the addition of metals or compositions to a fermentation reaction should be understood to include addition to either or both of these reactors. The mixture of components (including the culture and nutrient medium) found in the bioreactor is referred to as the broth or fermentation broth. The microorganism culture present in the fermentation broth is referred to as the broth culture and the broth culture density refers to the density of microorganism cells in the fermentation broth.

The process includes culturing, in a primary bioreactor containing a liquid nutrient medium, such as a solution added to the fermentation broth containing nutrients and other components appropriate for the growth of the microorganism culture, at least one strain of anaerobic, acetogenic bacteria capable of producing acetate from a gaseous substrate containing CO, CO₂ or H₂, or any mixture thereof, and supplying the gaseous substrate to the primary bioreactor. The fermentation process produces acetate. The acetate produced in the primary bioreactor is introduced to a secondary bioreactor containing a culture of at least one microalgae, capable of producing lipids from an acetate containing substrate.

The at least strain of anaerobic acetogenic bacteria capable of producing acetate from a gaseous substrate containing CO, CO₂ or H₂, or mixtures thereof are from the group consisting of Acetobacterium, Moorella, Clostridium, Pyrococcus, Eubacterium, Desulfobacterium, Cabroxydothermus, Acetogenium, Acetoanaerobium, Butyribaceterium, Peptostreptococcus, Ruminococcus, Oxobacter and Methanosarcina.

The bioreactors or fermenters include a fermentation device consisting of one or more vessels and/or towers or piping arrangements, which include the Continuous Stirred Tank Reactor (CSTR), Immobilized Cell Reactor (ICR), Trickle Bed Reactor (TBR), Moving Bed Biofilm Reactor (MBBR), Bubble Column, Gas Lift Fermenter, Membrane Reactor such as Hollow Fibre Membrane Bioreactor (HFMBR), Static Mixer, or other vessels or other device suitables for gas-liquid contact.

The primary bioreactor may be one or more reactors connected in series or parallel with a secondary bioreactor. The primary bioreactors house anaerobic fermentation to produce acids from a gaseous substrate. At least a portion of the acid product of the one or more primary bioreactors is used as a substrate in one or more secondary bioreactor(s). Similarly, the secondary bioreactor may encompass any number of further bioreactors that may be connected in series or in parallel with the primary bioreactor(s). Any one or more of these secondary bioreactors may also be connected to a further separator.

While the following description focuses on certain embodiments of the disclosure, the disclosure may be applicable to production of other alcohols and/or acids and the use of other substrates as will be known by persons of ordinary skill in the art to which the disclosure relates upon consideration of the present disclosure. Also, while particular mention is made to fermentation carried out using acetogenic microorganisms, the disclosure is also applicable to other micro-organisms which may be used in the same or different processes which may be used to produce useful products, including but not limited to other acids (including their corresponding conjugate bases) and alcohols.

### Fermentation Utilising a Gaseous Substrate.

An embodiment of the disclosure includes the production of acetic acid/acetate and ethanol from gaseous substrates comprising CO and optionally H₂ containing industrial flue gases. One such type of gas stream is tailgas from steel production plants, which typically contains 20-70% CO. Such gas streams may further comprise CO₂. Similar streams are produced from processing of any carbon-based feedstock, such as petroleum, coal, and biomass. Another embodiment of the disclosure includes the production of acetic acid/acetate from gaseous substrates comprising CO₂. H₂ may be part of the gaseous substrate or may be added to the gaseous substrate. The disclosure may also be applicable to reactions which produce alternative acids.

Processes to produce acetate and other alcohols from gaseous substrates are known. Exemplary processes include those described for example in WO2007/117157, WO2008/115080, U.S. Pat. Nos. 6,340,581, 6,136,577, 5,593,886, 5,807,722 and 5,821,111, each of which is incorporated herein by reference in their entirety.

Several anaerobic bacteria are known to be capable of carrying out the fermentation of CO to ethanol and acetic acid/acetate or the fermentation of CO₂ and H₂ to acetic acid/acetate and are suitable for use in the process of the present disclosure. Acetogens can convert gaseous substrates such as H₂, CO₂ and CO into products including acetic acid, ethanol and other fermentation products by the Wood-Ljungdahl pathway. Examples of such bacteria that are suitable for use in the disclosure include those of the genus Clostridium, such as strains of Clostridium ljungdahlii, including those described in WO 00/68407, EP 117309, U.S. Pat. Nos. 5,173,429, 5,593,886, and 6,368,819, WO 98/00558 and WO 02/08438, and Clostridium autoethanogenum (Abrini et al, Archives of Microbiology 161: pp 345-351). Other suitable bacteria include those of the genus Moorella, including Moorella sp HUC22-1, (Sakai et al, Biotechnology Letters 29: pp 1607-1612), and those of the genus Carboxydothermus (Svetlichny, V. A., Sokolova, T. G. et al (1991), Systematic and Applied Microbiology 14: 254-260). The disclosures of each of these publications are fully incorporated herein by reference. In addition, other acetogenic anaerobic bacteria may be selected for use in the process of the disclosure by a person of skill in the art. It will also be appreciated that a mixed culture of two or more bacteria may be used in the process of the present disclosure.

One exemplary micro-organism suitable for use in the present disclosure is Clostridium autoethanogenum that is available commercially from the Deutsche Sammlung von Mikroorganismen and Zellkulturen GmbH (DSMZ) and having the identifying characteristics of DSMZ deposit number DSMZ 10061.

The disclosure has further applicability to supporting the production of acetate from gaseous substrates comprising CO₂ and H₂. Acetobacterium woodii has been shown to produce acetate by fermentation of gaseous substrates comprising CO₂ and H₂. Buschhom et al. demonstrated the ability of A woodii to produce ethanol in a glucose fermentation with a phosphate limitation. One exemplary micro-organism suitable for use in the present disclosure is Acetobacterium woodii having the identifying characteristics of the strain deposited at the German Resource Centre for Biological Material (DSMZ) under the identifying deposit number DSM 1030.

Other suitable bacteria include those of the genus Moorella, including Moorella sp HUC22-1, (Sakai et al, Biotechnology Letters 29: pp 1607-1612), and those of the genus Carboxydothermus (Svetlichny, V. A., Sokolova, T. G. et al (1991), Systematic and Applied Microbiology 14: 254-260). Further examples include Morella thermoacetica, Moorella thermoautotrophica, Ruminococcus productus, Acetobacterium woodii, Eubacterium limosum, Butyribacterium methylotrophicum, Oxobacter pfennigii, Methanosarcina barkeri, Methanosarcina acetivorans, Desulfotomaculum kuznetsovii (Sipma et. al. Critical Reviews in Biotechnology, 2006 Vol. 26. Pp 41-65). In addition, it should be understood that other acetogenic anaerobic bacteria may be applicable to the present disclosure as would be understood by a person of skill in the art. It will also be appreciated that the disclosure may be applied to a mixed culture of two or more bacteria.

Culturing of the bacteria used in a process of the disclosure may be conducted using any number of processes known in the art for culturing and fermenting substrates using anaerobic bacteria. Exemplary techniques are provided in the “Examples” section below. In certain embodiments a culture of a bacterium of the disclosure is maintained in an aqueous culture medium. Preferably the aqueous culture medium is a minimal anaerobic microbial growth medium. Suitable media are known in the art and described for example in U.S. Pat. Nos. 5,173,429 and 5,593,886 and WO 02/08438, and in Klasson et al [(1992). Bioconversion of Synthesis Gas into Liquid or Gaseous Fuels. Enz. Microb. Technol. 14:602-608.], Najafpour and Younesi [(2006). Ethanol and acetate synthesis from waste gas using batch culture of Clostridium ljungdahlii. Enzyme and Microbial Technology, Volume 38, Issues 1-2, p. 223-228] and Lewis et al [(2002). Making the connection-conversion of biomass-generated producer gas to ethanol. Abst. Bioenergy, p. 2091-2094]. In particular embodiments of the disclosure, the minimal anaerobic microbial growth medium is as described hereinafter in the Examples section. By way of further example, those processes generally described in the following disclosures using gaseous substrates for fermentation may be utilised: WO98/00558, M. Demler and D. Weuster-Botz (2010). Reaction Engineering Analysis of Hydrogenotrophic Production of Acetic Acid by Acetobacterium woodii. Biotechnology and Bioengineering 2010; D. R. Martin, A. Misra and H. L. Drake (1985). Dissimilation of Carbon Monoxide to Acetic Acid by Glucose-Limited Cultures of Clostridium thermoaceticum. Applied and Environmental Microbiology, Vol 49, No. 6, pages 1412-1417.Typically, fermentation is carried out in any suitable bioreactor, such as a continuous stirred tank reactor (CTSR), a bubble column reactor (BCR) or a trickle bed reactor (TBR). Also, in some embodiments of the disclosure, the bioreactor may comprise a first, growth reactor in which the micro-organisms are cultured, and a second, fermentation reactor, to which fermentation broth from the growth reactor is fed and in which most of the fermentation product (ethanol and acetate) is produced.

### Feedstock

The carbon source for the fermentation can be a gaseous substrate comprising carbon monoxide optionally in combination with hydrogen, or a gaseous substrate comprising CO₂ optionally in combination with hydrogen or any combination thereof. For example, the gaseous substrate may be a CO and optionally H₂-containing, or a CO₂ and H₂-containing waste gas obtained as a by-product of an industrial process, or from some other source such as gasification.

As described above, the carbon source for the fermentation reaction is a gaseous substrate containing CO or CO₂ or both. The gaseous substrate may be a CO or CO₂-containing waste gas obtained as a by-product of an industrial process, or from some other source such as from automobile exhaust fumes or gasification. In some embodiments, the industrial process may be selected from ferrous metal products manufacturing, such as a steel mill, non-ferrous products manufacturing, petroleum refining processes, gasification of coal, electric power production, carbon black production, ammonia production, methanol production and coke manufacturing. In these embodiments, the CO-containing gas may be captured from the industrial process before it is emitted into the atmosphere, using any convenient method. Depending on the composition of the gaseous CO-containing substrate, it may also be desirable to treat it to remove any undesired impurities, such as dust particles before introducing it to the fermentation. For example, the gaseous substrate may be filtered or scrubbed using known methods.

In addition, it is often desirable to increase the CO or CO₂ concentration of a substrate stream (or partial pressure in a gaseous substrate) and thus increase the efficiency of fermentation reactions where CO or CO₂ is a substrate. Increasing CO or CO₂ partial pressure in a gaseous substrate increases mass transfer into a fermentation media. The composition of gas streams used to feed a fermentation reaction can have a significant impact on the efficiency and/or costs of that reaction. For example, O₂ may reduce the efficiency of an anaerobic fermentation process. Processing of unwanted or unnecessary gases in stages of a fermentation process before or after fermentation can increase the burden on such stages (e.g. where the gas stream is compressed before entering a bioreactor, unnecessary energy may be used to compress gases that are not needed in the fermentation). Accordingly, it may be desirable to treat substrate streams, particularly substrate streams derived from industrial sources, to remove unwanted components and increase the concentration of desirable components.

Hydrogen rich gas streams are produced by a variety of processes including steam reformation of hydrocarbons, and in particular steam reformation of natural gas. The partial oxidation of coal or hydrocarbons is also a source of hydrogen rich gas. Other sources of hydrogen rich gas include the electrolysis of water, by-products from electrolytic cells used to produce chlorine and from various refinery and chemical streams.

The gaseous substrate may further, or instead, comprise CO₂. Gas streams with high CO₂ content are derived from a variety of industrial processes and includes exhaust gasses from combustion of hydrocarbons, such as natural gas or oil. These processes include cement and lime production, and iron and steel production.

### Blending of Streams

In some embodiments, an industrial waste stream may be blended with one or more further streams to improve efficiency, acid and/or alcohol production and/or overall carbon capture of the fermentation reaction. Where industrial waste streams have a high CO or CO₂ content, but include minimal or no H₂, it may be desirable to blend one or more streams comprising H₂ with the industrial waste stream prior to introducing the blended stream to the fermenter. The overall efficiency, alcohol productivity and/or overall carbon capture of the fermentation will be dependent on the stoichiometry of the CO and H₂ or CO₂ and H₂ in the blended stream. In some embodiments, the blended stream may substantially comprise CO and H₂ the following molar ratios: at least 1:2 at least 1:4 or at least 1:6 or at least 1:8 or at least 1:10. In other embodiments the blended stream may comprise CO₂ and H₂ in the following molar ratios: at least 1:4 or at least 1:6 or at least 1:8 or at least 1:10.

Blending of streams may also have further advantages, such as where a waste stream comprising CO, CO₂ or H₂ is intermittent in nature. For example, an intermittent waste stream comprising CO and optionally H₂ may be blended with a substantially continuous stream comprising CO, CO₂, and/or H₂ and provided to the fermenter. In some embodiments, the composition and flow rate of the substantially continuous stream may be varied in accordance with the intermittent stream in order to maintain a substrate stream of substantially continuous composition and flow rate to the fermenter.

Blending two or more streams to achieve a desirable composition may involve varying flow rates of all streams, or one or more of the streams may be maintained constant while other stream(s) are varied in order to ‘trim’ or optimise the substrate stream to the desired composition. For streams that are processed continuously, little or no further treatment (such as buffering) may be necessary and the stream can be provided to the fermenter directly. However, it may be necessary to provide buffer storage for streams where one or more is available intermittently, and/or where streams are available continuously, but are used and/or produced at variable rates.

Monitoring the composition and flow rates of the streams prior to blending is advantageous. Control of the composition of the blended stream can be achieved by varying the proportions of the constituent streams to achieve a target or desirable composition. For example, a base load gas stream may be predominantly CO, and a secondary gas stream comprising a high concentration of H₂ may be blended to achieve a specified H₂:CO ratio. The composition and flow rate of the blended stream can be monitored by any means known in the art. The flow rate of the blended stream can be controlled independently of the blending operation; however the rates at which the individual constituent streams can be drawn must be controlled within limits. For example, a stream produced intermittently, drawn continuously from buffer storage, must be drawn at a rate such that buffer storage capacity is neither depleted nor filled to capacity.

At the point of blending, the individual constituent gases will enter a mixing chamber, which will typically be a small vessel, or a section of pipe. In such cases, the vessel or pipe may be provided with static mixing devices, such as baffles, arranged to promote turbulence and rapid homogenisation of the individual components. Buffer storage of the blended stream can also be provided if necessary, in order to maintain provision of a substantially continuous substrate stream to the bioreactor.

A processor adapted to monitor the composition and flow rates of the constituent streams and control the blending of the streams in appropriate proportions, to achieve the required or desirable blend may optionally be incorporated into the system. For example, particular components may be provided in an as required or an as available manner in order to optimise the efficiency of acetate productivity and/or overall carbon capture.

In certain embodiments of the disclosure, the system is adapted to continuously monitor the flow rates and compositions of at least two streams and combine them to produce a single blended substrate stream of optimal composition, and means for passing the optimised substrate stream to the fermenter.

By way of non limiting example, embodiments of the disclosure involve the utilization of CO gas from a steel production process. Typically, such streams contain little or no H₂, and it may be desirable to combine the stream comprising CO with a stream comprising H₂ in order to achieve a more desirable CO:H₂ ratio. H₂ is often produced in large quantities at a steel mill in the coke oven. A waste stream from the coke oven comprising H₂ can be blended with a steel mill waste stream comprising CO to achieve a desirable composition for fermentation.

Substrate streams derived from an industrial source are variable in composition. Furthermore, substrate streams derived from industrial sources comprising high CO concentrations (such as, for example, at least 40% CO, at least 50% CO or at least 65% CO) often have a low H2 component (such as less than 20% or less than 10% or substantially 0%). As such, it is particularly desirable that micro-organisms are capable of producing products by anaerobic fermentation of substrates comprising a range of CO and H₂ concentrations, particularly high CO concentrations and low H₂ concentrations. The bacteria of the present disclosure have a surprisingly high growth rate and acetate production rate while fermenting a substrate comprising CO (and no H₂).

The processes of the present disclosure can be used to reduce the total atmospheric carbon emissions from industrial processes, by capturing CO-containing gases or CO₂-containing gasses produced as a result of such processes and using them as substrates for the fermentation processes described herein.

Alternatively, in other embodiments of the disclosure, the CO-containing gaseous substrate may be sourced from the gasification of biomass. The process of gasification involves partial combustion of biomass in a restricted supply of air or oxygen. The resultant gas typically comprises mainly CO and H₂, with minimal volumes of CO₂, methane, ethylene and ethane. For example, biomass by-products obtained during the extraction and processing of foodstuffs such as sugar from sugarcane, or starch from maize or grains, or non-food biomass waste generated by the forestry industry may be gasified to produce a CO-containing gas suitable for use in the present disclosure.

The CO-containing gaseous substrate may contain a major proportion of CO. In particular embodiments, the gaseous substrate comprises at least about 25%, at least about 30%, at least about 40%, at least about 50%, at least about 65%, or at least about 70% to about 95% CO by volume. It is not necessary for the gaseous substrate to contain any hydrogen. The gaseous substrate also optionally contain CO₂, such as about 1% to about 30% by volume, such as about 5% to about 10% CO₂.

### Reaction Stoichiometry

Anaerobic bacteria have been demonstrated to produce ethanol and acetic acid from CO, CO₂ and H₂ via the Acetyl-CoA biochemical pathway. The stoichiometry for the formation of acetate from a substrate comprising CO by acetogenic microorganisms is as follows:

4CO+2H₂O→CH₃COOH+2CO₂

And in the presence of H₂:

4CO+4→H₂→2CH₃COOH

Anaerobic bacteria have also been demonstrated to produce acetic acid from CO₂ and H₂. The stoichiometry for the formation of acetate from a substrate comprising CO₂ and H₂ by acetogenic bacteria including Acetobacterium woodii:

4H₂+2CO₂→CH₃COOH+2H₂O

It will be appreciated that for growth of the bacteria and fermentation to occur, in addition to the CO, CO₂ and/or H₂-containing substrate gas, a suitable liquid nutrient medium will need to be fed to the bioreactor. A nutrient medium will contain vitamins and minerals sufficient to permit growth of the micro-organism used. Anaerobic media suitable for the fermentation of ethanol using CO as the sole carbon source are known in the art. For example, suitable media are described in U.S. Pat. Nos. 5,173,429 and 5,593,886 and WO 02/08438 as well as other publications referred to above.

The fermentation is carried out under appropriate conditions for the CO, or CO₂ and H₂-to-acetate fermentation to occur. Reaction conditions that should be considered include pressure, temperature, gas flow rate, liquid flow rate, media pH, media redox potential, agitation rate (if using a continuous stirred tank reactor), inoculum level, maximum gas substrate concentrations to ensure that CO or CO₂ in the liquid phase does not become limiting, and maximum product concentrations to avoid product inhibition.

In one embodiment, the fermentation is carried out at a temperature of about 34° C. to about 37° C. In one embodiment, the fermentation is carried out at a temperature of about 34° C. The inventors note that this temperature range may assist in supporting or increasing the efficiency of fermentation including, for example, maintaining or increasing the growth rate of bacteria, extending the period of growth of bacteria, maintaining or increasing production of metabolites (including acetate), maintaining or increasing CO or CO₂ uptake or consumption.

Specific reaction conditions will depend partly on the microorganism used. However, in general, the fermentation may be performed at pressure higher than ambient pressure. Operating at increased pressures allows a significant increase in the rate of CO and/or CO₂ transfer from the gas phase to the liquid phase where it can be taken up by the micro-organism as a carbon source to produce acetate. This in turn means that the retention time (defined as the liquid volume in the bioreactor divided by the input gas flow rate) can be reduced when bioreactors are maintained at elevated pressure rather than atmospheric pressure.

Also, since a given CO, or CO₂ and H₂-to-acetate conversion rate is in part a function of the substrate retention time and achieving a desired retention time in turn dictates the required volume of a bioreactor, the use of pressurized systems can greatly reduce the volume of the bioreactor required, and consequently the capital cost of the fermentation equipment.

### Microalgae Utilizing Acids as a Substrate for Lipid Production

The disclosure has applicability to supporting the production of lipids from acetate containing substrates. One such type of substrate is acetate derived from the conversion of gaseous substrates comprising CO, or CO₂ and H₂, or mixtures thereof by anaerobic microbial fermentation.

A number of microorganisms are known to be capable of carrying out the fermentation of sugars to lipids and are suitable for use in the process of the present disclosure. For ease of understanding, these microorganisms will be referred to as microalgae. Examples of such microalgae are those of the genus Schizochytrium or Scenedesmus.

Microalgae have been shown to produce lipids by heterotrophic fermentation of substrates comprising acetate (Huang, G, Chen, F., Wei, D., Zhang, X., Chen, G. “Biodiesel production by microalgal biotechnology” Applied Energy, Vol 87(1), 2010, 38-46; Ren, H., Liu, B., Ma, C., Zhao, L., Ren, N. “A new lipid-rich microalga Scenedesmus sp. strain R-16 isolated using Nile red staining: effects carbon and nitrogen sources and initial pH on the biomass and lipid production. Biotechnology for Biofuels, 2013, 6(143)). Production of lipid(s) by microalgae may be improved by a nitrogen limitation. For example, a carbon to nitrogen ratio of 49:1 has a significant impact on lipid production.

Suitable microalgae for use in the process of the disclosure include those of the genus Chlorella, Chlamamydomonas, Dunaliella, Euglena, Parvum, Tetraselmis, Porphyridium, Spirulina, Synechoccus, Anabaena, Schizochytrium, Botyrococcus, Fucus, Parachlorella, Pseudochlorella, Brateococcus, Prototheca and Scenedesmus, Thraustochytrium, Japonochytrium, Aplanochytrium, Elina, and Labyrinthula (or Labyrinthuloides or Labyrinthulomyxa). In a one embodiment, the microalgae is a thraustochytrid of the genus Thraustochytrium. The thraustochytrid may be any species of Thraustochytrium including, but not limited to, the thraustrochytrids described in Gupta, Biotechnol Adv, 30: 1733-1745 (2012) or Gupta, Biochem Eng J, 78: 11-17 (2013).

Other microalgae may be applicable in the present disclosure as would be understood by a person of skill in the art. The disclosure may also be applied to a mixed culture of two or more microalgae species. Culturing of the microalgae used in a method of the disclosure may be conducted using any number of processes known in the art. The conversion process is carried out in any suitable bioreactor as discussed above. In certain embodiments, the microalgae bioreactor will require an oxygen or air inlet for growth of the microalgae.

The microalgae contained within the secondary bioreactor are capable of converting acetate to lipids, wherein the lipids accumulate within the membrane fraction of the biomass. Following lipid accumulation, the biomass of the secondary bioreactor can be passed to an extraction system. The extraction system may be utilized for extraction of accumulated lipids from the membrane fraction of the microalgae biomass. Lipid extraction may be conducted using any number of processes known in the art.

The lipids produced may be further processed to provide chemicals, fuels or fuel components such as, for example, hydrocarbons, hydrogenation-derived renewable diesel (HDRD), fatty acid methyl esters (FAME), fatty acid ethyl esters (FAEE), and biodiesel by means known in the art. Various derivatives, such as cleaning and personal care products, use components such as surfactants, fatty alcohols, and fatty acids, all of which lipids may be provided as a substitute. Further, various oleochemicals can be produced from lipids. Omega-3 fatty acid is one or more of alpha-linolenic acid (ALA), eicosapentaenoic acid (EPA), and docosahexaenoic acid (DHA).

### Media Recycle

The efficiency of the fermentation processes of the disclosure can be further improved by recycling a media-containing stream exiting the secondary bioreactor to at least one primary reactor. The media-containing stream exiting the secondary bioreactor may contain unused metals, salts and other nutrient components. Recycling the media-containing exit stream to a primary reactor reduces the cost of providing a continuous nutrient media to the primary reactor. Recycling has the further benefit of reducing the overall water requirements of the continuous fermentation process. The media containing stream exiting the bioreactor can be treated before being passed back to a primary reactor.

Recycling the media-containing stream is further beneficial as it helps reduce pH control costs in the primary bioreactor. Accumulation of acetate product results in a lowering of the pH of the broth in the primary bioreactor, which is harmful to the culture suspended in the media. With acetate accumulation in the primary bioreactor, a base, such as NH₃ or NaOH, must be added to the media to increase the pH in the primary bioreactor. By passing broth to the secondary bioreactor and then recycling the media-containing stream back to the primary bioreactor, the microalgae of the secondary bioreactor consume the acetate and increase the pH of the media-containing stream recycled to the primary bioreactor. With acetate being removed from the system in the secondary bioreactor, the need for expensive bases for pH adjustment to the media of the primary reactor is reduced or eliminated.

The biomass is then separated from the secondary bioreactor fermentation broth and processed to recover one or more lipid products. The remainder of the broth, after separation of the biomass, may then be recycled to a primary reactor. The biomass should be removed to result in an amount of biomass that is less than 20 mass-% of the recycle stream, measured as grams on a dry cell basis per liter of solution, or less than less than 10 mass-% of the recycle stream, measured as grams on a dry cell basis per liter of solution, or less than 5 mass-% of the recycle stream, measured as grams on a dry cell basis per liter of solution. The recycle stream can be further treated to remove soluble proteins or other unwanted components prior to being passed to the primary reactor. Additional metals and salts can be added to the recycle stream before returning to the primary reactor to provide nutrients at a desired composition. The pH of the stream may be monitored and adjusted according to the needs of the fermentation process of the primary bioreactor.

As the microalgae require oxygen for growth in the secondary bioreactor, any media recycled back to the primary bioreactor will need to have oxygen substantially removed, as oxygen present in the primary bioreactor is harmful to the anaerobic culture. Therefore, the broth stream exiting the secondary bioreactor may be passed through an oxygen scrubber to remove oxygen prior to being passed to the primary reactor. Oxygen should be removed to an amount less than 5 mole-%, less than 2 mole-% less than 0.5 mole-%, or less than 0.001 mole-% of the stream recycled to the primary bioreactor.

### Tail Gas Recycle

As discussed above, the conversion of acetate to lipids in the secondary bioreactor forms CO₂. Lipid formation from acetate follows the stoichiometric conversion which always leads to the co-formation of CO₂. One example includes:

27CH₃CO₂H+5O₂→2C₁₈H₃₀O₂+18CO₂+24H₂O

In this example, the formation of linolenic acid shows that for every 2 carbon atoms contained in the lipid molecule made from acetate, 1 carbon atom will be released as CO₂ resulting in 33% of the carbon contained in the in initial acetate feed is lost as CO₂. Other fatty acids may be produced by the microorganism, but the ratio of carbon captured, and CO₂ released remains about the same.

This disclosure provides for recycling the tail gas from the secondary bioreactor to the primary reactor where the CO₂ may be used as substrate. One challenge however is that the secondary bioreactor operates in an aerobic mode while the primary bioreactor operates in an anaerobic mode. Therefore, O₂ present in the tail gas from the secondary bioreactor must be separated and removed from the CO₂ containing tail gas stream before the tail gas is recycled to the primary bioreactor. The amount of O₂ removed should be enough so that any remaining O₂ that is passed to the primary bioreactor does not adversely affect the operations of the primary bioreactor beyond tolerances. Suitable techniques for separation include pressure swing adsorption (PSA), membrane separation, acid gas removal techniques, CO₂ solvents such as amine, methanol and the like for absorption using a solvent, and scrubbing with a basic solution. The separation step may also involve the separation of nitrogen. The separation step may involve two or more separation stages in series or in parallel. The separation stages may be of the same technique or may employ different techniques. For example, two PSA units in series may be employed. In an embodiment, a first PSA may be used to remove CO₂ while a second PSA may be used to remove O₂ and reject N₂. The separated oxygen may be returned to the secondary bioreactor.

The secondary bioreactor may be operated in an oxygen limited mode in order to deplete the amount of unreacted O₂ in the tail gas. Alternatively, in embodiment where it is desirable to have residual O₂ in the secondary bioreactor, the tail gas may be sparged through yet another vessel that is continuously fed by and recycled to the secondary bioreactor. The microorganisms in the “another” vessel are controlled to be under oxygen limited conditions to achieve the removal of O₂.

A further advantage provided by this disclose is with the incorporation of environmentally friendly produced H₂ which is used in the process. Environmentally friendly H₂ is produced by one of two ways. The first comes from steam reforming of biogenic methane produced by methanogenic bacteria from agricultural and food wastes. The second comes from the usage of a water electrolyzer that provides H₂ for the anaerobic stage of the fermentation process and, in a separated stream, neat oxygen for the aerobic stage of the fermentation process. As H₂ is needed in the primary bioreactor, and O₂ is needed in the secondary bioreactor, the water electrolyzer may be used to provide the H₂ and O₂ needed for both the fermentation stages thereby providing lipid production from H₂ and CO₂ in the most environmentally friendly way without the addition of atmospheric nitrogen.

Furthermore, electing to employ O₂ generated by a water electrolyzer as opposed to, for example, air, has the advantage that no unnecessary inert gas is entering the system. This is important since the tail gas, including CO₂, produced in the secondary bioreactor is recycled to the fist reactor, an inert gas portion of the tail gas would accumulate over time in the system. Some additional mechanism would become necessary to remove the accumulating inert gas. Neat O₂ as a feed gas also helps to improve O₂ mass transfer according to its partial pressure.

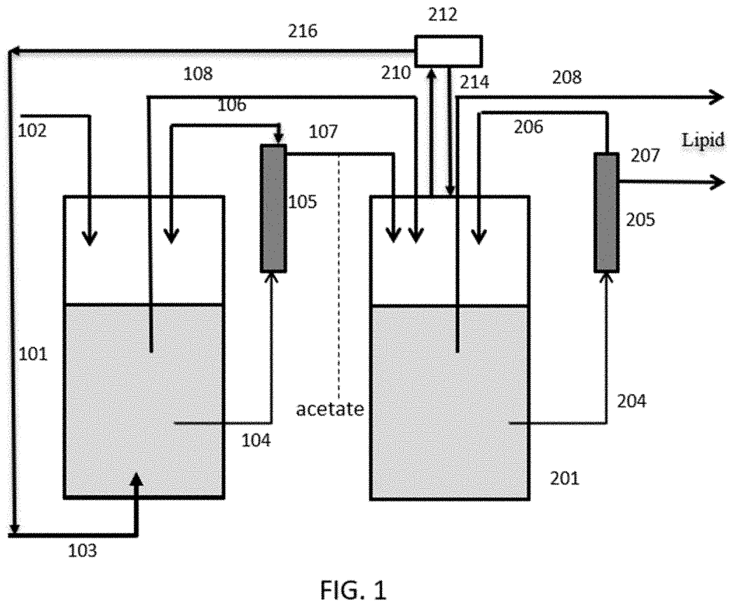

The methods and systems of the disclosure are herein described with reference to the Figures. FIG. 1 demonstrates a two-stage system to produce lipid(s) from a gaseous stream comprising CO and H₂, or CO₂ and H₂. The system provides a primary bioreactor 101 having a media inlet 102, a gas inlet port 103, a separator means 104, a permeate stream outlet 107, and a bleed stream outlet 108. The primary bioreactor is connected to a secondary bioreactor 201, having a separator 205, a permeate stream outlet 207 and a bleed stream outlet 208.

In use, the primary bioreactor 101 contains fermentation broth comprising a culture of one or more acetogenic bacteria in a liquid nutrient medium. Medium is added to the bioreactor 101 in a continuous or semi-continuous manner throughout the media inlet 102. A gaseous substrate is supplied to the bioreactor 101 via the gas inlet port 103. The separator means is adapted to receive at least a portion of broth from the bioreactor 101 via a first output conduit 104 and pass it through the separator 105 configured to substantially separate the microorganism cells (the retentate) from the rest of the fermentation broth (the permeate). At least a portion of the retentate is returned to the first bioreactor via a first return conduit 106 which ensures that the broth culture density is maintained at an optimal level. The separator 105 is adapted to pass at least a portion of the permeate out of the bioreactor 101 via a permeate delivery conduit 107. The permeate delivery conduit 107 feeds the cell free permeate to the secondary bioreactor 201. In certain embodiments of the disclosure, at least a portion of the cell free permeate is removed for product extraction and/or at least a portion of the cell free permeate is recycled to primary bioreactor with the remainder of the cell free permeate stream being fed to the secondary bioreactor 201. A broth bleed output 108 is provided to directly feed broth from the primary bioreactor 101 to the secondary bioreactor 202. In certain embodiments the broth bleed and permeate are combined prior to being fed to the secondary bioreactor.

Secondary bioreactor 201 contains a culture of one more microalgae in a liquid nutrient medium. A microalgae is used as a specific example, and any suitable microorganism may be employed in secondary bioreactor 201. Secondary bioreactor 201 receives broth and/or permeate from primary bioreactor 101 in a continuous or semi-continuous manner through broth bleed output 108 and permeate delivery conduit 107. The separator 205 is adapted to receive at least a portion of broth from secondary bioreactor 201 via a first output conduit 204. Separator 205 is configured to substantially separate the microorganism cells (the retentate) from the rest of the fermentation broth (the permeate). At least a portion of the retentate is returned to secondary bioreactor 201 via a second return conduit 206 which ensures that the broth culture density in secondary bioreactor 201 is maintained at an optimal level. Separator 205 is adapted to pass at least a portion of the permeate out of secondary bioreactor 201 via a permeate removal conduit 207. Broth bleed output 208 is provided to directly remove broth from secondary bioreactor 201. Both bleed output 208 may be treated to remove the biomass for lipid extraction using known methods. The substantially biomass free bleed stream and the permeate streams may be combined to produce a combined stream. In certain aspects of the disclosure, the combined stream can be returned to the primary reactor to supplement the liquid nutrient medium being continuously added. In certain embodiments it may be desirable to further process the recycle stream to remove undesired by products of the secondary fermentation. In certain embodiments, the pH of the recycle stream may be adjusted, and further vitamins and/or metals added to supplement the stream.

Tail gas stream 210 from secondary bioreactor 201 is passed to oxygen removal unit 212 to generate a substantially oxygen free CO₂ stream. Oxygen removal unit may be, for example, one or more PSA units, membrane separation units, scrubbers, absorption using solvent(s), or any combination thereof. Substantially oxygen free CO₂ stream is passed in line 216 and recycled to primary bioreactor 101 through inlet gas port 103. Substantially oxygen free means the stream comprises less than about 1 mol-percent O₂, less than about 500 mol-ppm O₂ or less than about 100 mol-ppm O₂. O₂ that is removed from the tail gas 210 in oxygen removal unit 212, may be recycled to the secondary bioreactor 201 through line 214.

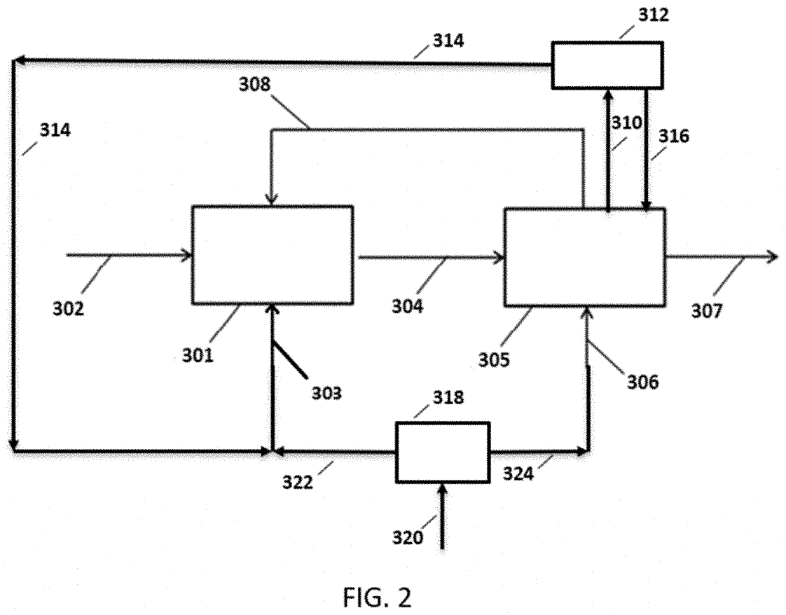

FIG. 2 demonstrates a simplified system for the production of lipid(s) from a gaseous stream comprising CO and H₂, or CO₂ and H₂, wherein the substantially acetate-free media is recycled from the secondary bioreactor to the primary bioreactor. The system includes a primary anaerobic bioreactor 301 having a media inlet 302, a gas inlet port 303, an acetate-containing treated bleed stream 304, a secondary aerobic bioreactor 305, an oxygen source 306, a lipid and biomass-containing product stream 307, and an acetate-depleted recycled media stream.

In use, the primary bioreactor 301 contains fermentation broth comprising a culture of one or more acetogenic bacteria in a liquid nutrient medium. Media is added to primary bioreactor 301 through the media inlet 302. A gaseous substrate comprising either CO and optionally H₂, or CO₂ and H₂, or mixtures thereof, is supplied to the primary bioreactor 301 via a gas inlet port 303, wherein the gas is converted by the bacteria to acetate. The primary bioreactor 301 is maintained at a pH in the range of 2.5-5, or 3-4, or 6.5-7, with pH optionally partially controlled by the addition of base as necessary. The acetate product leaves the primary bioreactor in an aqueous broth stream which is treated to remove the biomass using known methods. The resulting acetate-containing treated bleed stream 304 is fed to a secondary aerobic bioreactor 305. In the secondary bioreactor 305, acetate in the treated bleed stream is converted to lipids and non-lipid biomass by a microorganism, such as for example a microalgae. Oxygen is supplied to the aerobic fermentation by an oxygen or air inlet port 306. The lipid-containing microalgae cells are removed from the secondary bioreactor 305 by filtration, resulting in a lipid and biomass containing product stream 307 and a permeate stream 308. Because the aerobic fermentation consumes acetate, the pH of the broth increases as acetate is consumed, and the pH of the permeate stream 308 is consequently nominally higher than the pH of the acetate-containing broth stream 304. The dilution rate of the secondary bioreactor 305 is maintained such that the pH of the permeate stream 308 remains in the range of, for example, 5-7; or 7.0-7.5; or 7.5-9; or 10-11. The acetate-depleted permeate stream 308 is returned to the primary bioreactor 301. In addition to recycling a substantial portion of the water, salts, metals, and other nutrients that make up the media of the primary bioreactor 301, the recycled permeate stream 308 acts to significantly reducing the cost of fermentation pH control relative to a system in which pH is controlled only by direct addition of base to the bioreactor media.

Tail gas generated in secondary bioreactor 305 is removed in tail gas line 310 and conducted to oxygen separation unit 312. Oxygen separation unit may be, for example, one or more PSA units, membrane separation units, scrubbers, absorption using solvent(s), or any combination thereof. At least oxygen is removed from the tail gas in the oxygen separation unit. The resulting CO₂ stream that is substantially free of O₂ is then conducted in line 314 to gas inlet port 303 and introduced to primary bioreactor 301. O₂ removed in the oxygen separation unit may be recycled to secondary bioreactor 305 through line 316. FIG. 2 further illustrates the embodiment employing the optional water electrolyzer. Water stream 320 is introduced into electrolyzer 318 where a H₂ stream is generated by electrolysis and the H₂ stream is conducted in line 322 to gas inlet port 303 and introduced into primary bioreactor 301 along with the gaseous substrate discussed above. Similarly, electrolyzer 318 generates O₂ stream which is conducted in line 324 to inlet port 306 of secondary bioreactor 305.

### EXAMPLES

### Materials and Methods

### Media:

Solution A

| | | | | |
|---|---|---|---|---|
| NH₄Ac | 3.083 | g | KCl | 0.15 g |
| MgCl₂•6H₂O | 0.4 | g | NaCl (optional) | 0.12 g |
| CaCl₂•2H₂O | 0.294 | g | Distilled Water | Up to 1L |

Solution B

| | | | |
|---|---|---|---|
| Biotin | 20.0 mg | Calcium D-(*)- | 50.0 mg |
| | | pantothenate | |
| Folic acid | 20.0 mg | Vitamin B12 | 50.0 mg |
| Pyridoxine•HCl | 10.0 mg | p-Aminobenzoic acid | 50.0 mg |
| Thiamine•HCl | 50.0 mg | Thioctic acid | 50.0 mg |
| Riboflavin | 50.0 mg | Distilled water | To 1 Litre |
| Nicotinic acid | 50.0 mg | | |

Solution C

| Component/0.1M | | | Component/0.1M | | |
|---|---|---|---|---|---|
| solution (aq) | Quantity/ml | | solution (aq) | Quantity/ml | |
| FeCl₃ | 10 | ml | Na₂MoO₄ | 0.1 | ml |
| CoCl₂ | 1 | ml | ZnCl₂ | 1 | ml |
| NiCl₂ | 0.1 | ml | Na₂WO₄ | 0.1 | ml |

| | | | | |
|---|---|---|---|---|
| H₃BO₃ | 1 | ml | Distilled water | To 1 Litre |

Solution D

| Component | Conc. (in H₂O) | | Component | Conc. (in H₂O) |
|---|---|---|---|---|
| FeSO₄ | 0.1 | mol/L | Na₂MoO₄ | 0.01 mol/L |
| CoCl₂ | 0.05 | mol/L | ZnCl₂ | 0.01 mol/L |
| NiCl₂ | 0.05 | mol/L | H₃BO3 | 0.01 mol/L |

Solution E

| Media Component | Quantity | | Media Component | Quantity | |
|---|---|---|---|---|---|
| MgCl₂•6H₂O | 0.5 | g | Solution B | 10 | mL |
| NaCl | 0.2 | g | Solution C | 10 | mL |
| CaCl₂ | 0.2 | g | Resazurin (2 mg/L | 1 | mL |
| | | | stock) | | |
| NaH₂PO₄ | 2.04 | g | FeCl₃ | 1 | ml |
| NH₄Cl | 2.5 | g | Cysteine•HCl | 0.5 | g |
| | | | monohydrate | | |
| 85% H₃PO₄ | 0.05 | ml | Distilled water | To 1 litre | |
| KCl | 0.15 | g | | | |

Solution F

| Component | Quantity | Component | Quantity |
|---|---|---|---|
| FeCl₃ (0.1M solution) | 30 ml | Na₂S•9H₂0 | 2.8 g |
| CoCl₂ (0.1M solution) | 30 ml | Distilled water | To 300 ml |
| NiCl₂ (0.1M solution) | 30 ml | | |

The two types of Clostridium autoethanogenum used were those deposited at the German Resource Centre for Biological Material (DSMZ) and allocated the accession numbers DSM 19630 and DSM 23693. DSM 23693 was developed from Clostridium autoethanogenum strain DSM19630 (DSMZ, Germany) via an iterative selection process.

Bacteria: Acetobacterium woodii were obtained from the German Resource Centre for Biological Material (DSMZ). The accession number given to the bacteria is DSM 1030.

Preparation of Na₂S. A 500 ml flask was charged with Na₂S (93.7 g, 0.39 mol) and 200 ml H₂O. The solution was stirred until the salt had dissolved and sulfur (25 g, 0.1 mol) was added under constant N₂ flow. After 2 hours stirring at room temperature, the “Na₂Sₓ” solution (approx 4M with respect to [Na] and approx 5M with respect to sulfur), now a clear reddish brown liquid, was transferred into N₂ purged serum bottles, wrapped in aluminum foil.

Preparation of Cr (II) solution—A 1 L three necked flask was fitted with a gas tight inlet and outlet to allow working under inert gas and subsequent transfer of the desired product into a suitable storage flask. The flask was charged with CrCl₃.6H₂0 (40 g, 0.15 mol), zinc granules [20 mesh] (18.3 g, 0.28 mol), mercury (13.55 g, 1 mL, 0.0676 mol) and 500 ml of distilled water. Following flushing with N₂ for one hour, the mixture was warmed to about 80° C. to initiate the reaction. Following two hours of stirring under a constant N₂ flow, the mixture was cooled to room temperature and continuously stirred for another 48 hours by which time the reaction mixture had turned to a deep blue solution. The solution was transferred into N₂ purged serum bottles and stored in the fridge for future use.

### Sampling and Analytical Procedures

Media samples were taken at intervals over a 30 day period

All samples were used to establish the absorbance at 600 nm (spectrophotometer) and the level of substrates and products (GC or HPLC). HPLC was routinely used to quantify the level of acetate.

HPLC: HPLC System Agilent 1100 Series. Mobile Phase: 0.0025N Sulfuric Acid. Flow and pressure: 0.800 mL/min. Column: Alltech IOA; Catalog #9648, 150×6.5 mm, particle size 5 μm. Temperature of column: 60° C. Detector: Refractive Index. Temperature of detector: 45° C.

Method for sample preparation: 400 μL of sample and 50 μL of 0.15M ZnSO₄ and 50 μL of 0.15M Ba(OH)₂ are loaded into an Eppendorf tube. The tubes are centrifuged for 10 min. at 12,000 rpm, 4° C. 200 μL of the supernatant are transferred into an HPLC vial, and 5 μL are injected into the HPLC instrument.

Headspace Analysis: Measurements were carried out on a Varian CP-4900 micro GC with two installed channels. Channel 1 was a 10 m Mol-sieve column running at 70° C., 200 kPa argon and a backflush time of 4.2 s, while channel 2 was a 10 m PPQ column running at 90° C., 150 kPa helium and no backflush. The injector temperature for both channels was 70° C. Runtimes were set to 120 s, but all peaks of interest would usually elute before 100 s. Headspace of fermenter was analysed automatically by Gas-GC (Varian 4900 Micro-GC) at hourly.

Cell Density: Cell density was determined by counting bacterial cells in a defined aliquot of fermentation broth. Alternatively, the absorbance of the samples was measured at 600 nm (spectrophotometer) and the dry mass determined via calculation according to published procedures.

### Example 1: Fermentation of CO in Bioreactor to Produce Acetate

A glycerol stock of a Clostridium autoethanogenum was revived in serum bottles. The glycerol stock stored at 80° C. was slowly thawed and transferred into the serum bottle using a syringe. This process was carried out inside an anaerobic chamber. The inoculated serum bottle was subsequently removed from the anaerobic chamber and pressurized to a total of 45 psi using a CO-containing gas mixture (40% CO, 3% H₂, 21% CO₂, 36% N₂). The bottle was then placed horizontally on a shaker inside an incubator at a temperature of 37 C. After two days of incubation and after verifying that the culture grew, the bottle was used to inoculate another set of eight gas containing serum bottles with 5˜mL of this culture. These serum bottles were incubated for another day as described above and then used to inoculate 5 L of liquid medium that was prepared in a 10 L CSTR. The initial CO containing gas flow was set at 100˜mL/min and the stirring rate was set to a low 200 rpm. When the microbes started consuming gas the agitation and gas flows were increased to 400 rpm and 550 mL/min. After two days of growth in batch mode, the fermenter was turned continuous with a dilution rate of 0.25 l/day. The dilution rate was increased by 0.25 l/day to a value of 1 l/day every 24˜h.

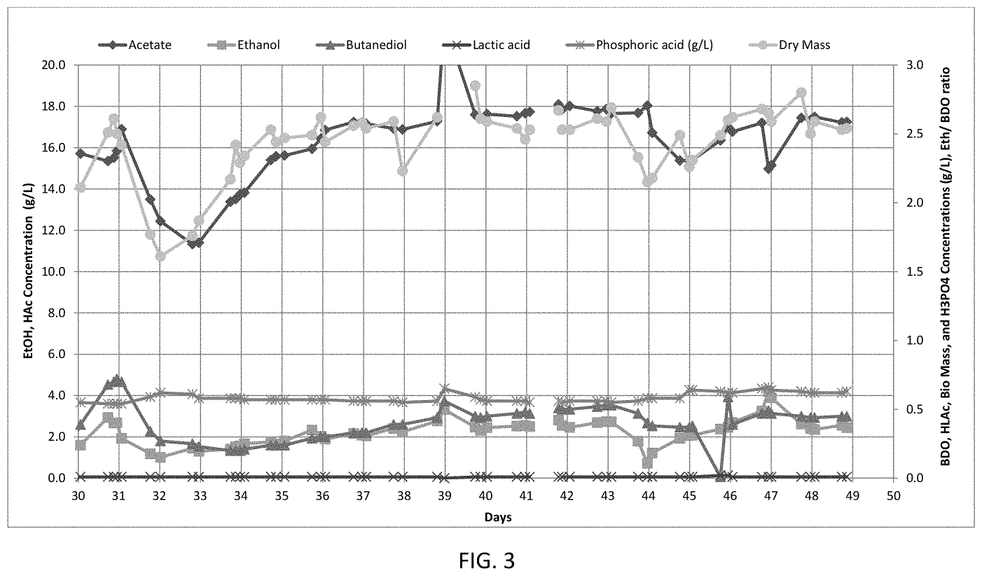

Metabolite and microbial growth can be seen in FIG. 3. Acetate concentration in the reactor carried from between around 10 g/L to over 20 g/L. The dilution rate was 1.0. Productivity rates for acetate were between 10 g/L/day and over 20 g/L/day. The ratio of acetate to ethanol varied between around 5:1 to around 18:1.

### Example 2: Fermentation of CO₂ and H₂ in Bioreactor to Produce Acetate

Media at pH 6.5 was prepared as using the protocol defined by Balch et al (See, e.g., Balch et al, (1977) International Journal of Systemic Bacteriology, 27:355-361). A three-litre reactor was filled with 1500 ml of the media. Oxygen was removed from the media by continuously sparging with N₂. The gas was switched from N₂ to a mixture of 60% H₂, 20% CO₂, and 20% N₂ 30 minutes before inoculation. The inoculum (150 ml) came from a continuous Acetobacterium woodii culture fed with the same gas mixture. The bioreactor was maintained at 30° C. and stirred at 200 rpm at the time of inoculation. During the following batch growth phase, agitation was increased incrementally to 600 rpm. The gas flow was increased incrementally by 50 ml/min according to the dropping H₂/CO₂ in the headspace as a result of the increasing biomass. To compensate for the acetic acid produced, the pH was automatically controlled to 7 using 5 M NaOH. Throughout the fermentation, a 0.5M solution of Na2S was pumped into the fermenter at a rate of 0.2 ml/hour. The culture was made continuous after 1 day. To reach high biomass along with high gas consumption, it is necessary to keep the acetate concentration in the fermenter at levels below 20 g/L. This was realized by running the fermenter at a relatively high dilution rate (D˜1.7/day) while retaining the microbes in the fermenter with a polysulfone membrane filtration system with 0.1 μm pore size (GE healthcare hallow fibre membrane). The medium for the continuous culture was solution A excluding the composite trace metal solution, which was fed separately at a rate of 1.5 ml/hour using an automated syringe pump. The medium was degassed at least 1 day before and continuously degassing throughout fermentation process.

### Results

Over a thirty-day period acetate is produced at a concentration of 12.5 g/L. The productivity rate of acetate averaged at 21.8 g/L per day.

The maximum concentration of acetic acid in a continuous culture was 17.76 g/L (296 mM)

### Example 3 Microalgae Acetate Utilisation

It has recently been demonstrated that the microalgae may utilise acetate as a carbon source for production of lipids. Ren et al. have shown that Scenedesmus sp. cultured in a liquid medium comprising acetate as a carbon source produced a total lipid content 43.4% and a maximum biomass concentration of 1.86 g L⁻¹ (Ren, H., Liu, B., Ma, C., Zhao, L., Ren, N. “A new lipid-rich microalga Scenedesmus sp. strain R-16 isolated using Nile red staining: effects carbon and nitrogen sources and initial pH on the biomass and lipid production. Biotechnology for Biofuels, 2013, 6(143)).

In the present system, acetate derived from anaerobic fermentation of a gaseous substrate is fed to a CSTR comprising microalgae, such as Scenedesmus sp. The microalgae is cultured in media at 25° C and a pH of 7. Agitation is set at 150 rpm. A nitrogen source, such as sodium nitrate, should also be present in the media at a range between 0.1-1.0 g/L, depending on the acetate concentration. Once all acetate in the media is converted to biomass, lipids are extracted from the biomass using known extraction methods.

The reference to any prior art in this specification is not, and should not be taken as, an acknowledgement or any form of suggestion that that prior art forms part of the common general knowledge in the field of endeavour in any country.

Throughout this specification and any claims which follow, unless the context requires otherwise, the words “comprise”, “comprising” and the like, are to be construed in an inclusive sense as opposed to an exclusive sense, that is to say, in the sense of “including, but not limited to”.

1. A method for producing at least one lipid product from CO₂ and H₂, the method comprising:
i. receiving a gaseous substrate comprising at least CO₂ and H₂ in a first bioreactor containing a culture of at least one first microorganism in a first liquid nutrient medium and fermenting the gaseous substrate to produce acetate product in a first fermentation broth;
ii. passing at least a portion of the first fermentation broth to a second bioreactor containing a culture of at least one second microorganism in a second liquid nutrient medium, where the second microorganism is not the same as the first microorganism and is selected from Scenedesmus, Thraustochytrium, Japonochytrium, Aplanochytrium, Elina, and Labyrinthula, and fermenting the acetate product to produce at least one lipid product in a second fermentation broth;
iii. obtaining a tail gas comprising at least CO₂ and O₂ from the second bioreactor; and
iv. separating at least a portion of the O₂ from the tail gas and recycling at least a portion of the reminder of the tail gas to the first bioreactor.

2. The method of claim 1, wherein the production rate of acetate in the first bioreactor is at least 10 g/L/day.

3. The method of claim 1, wherein at least one of the first microorganisms in the first bioreactor is selected from Acetobacterium, Moorella, Clostridium, Pyrococcus, Eubacterium, Desulfobacterium, Carboxydothermus Acetogenium, Acetoanaerobium, Butyribacterium, Peptostreptococcus, Ruminococcus, Oxobacter, and Methanosarcina.

4. The method of claim 1 wherein at least one of the first micoorganisms in the first bioreactor is Acetobacterium woodii.

5. The method of claim 1, wherein the at least one of the second microorganisms in the second bioreactor is Thraustochytrium.

6. The method of claim 1, further comprising producing, from the at least one lipid product, at least one tertiary product selected from hydrogenation-derived renewable diesel, fatty acid methyl esters, fatty acid ethyl esters, and biodiesel.

7. The method of claim 1, further comprising limiting at least one nutrient in the second liquid nutrient medium in the second bioreactor to increase lipid production.

8. The method of claim 7, wherein the limited nutrient is nitrogen.

9. The method of claim 1, wherein the at least one lipid product is a polyunsaturated fatty acid.

10. The method of claim 9, wherein the polyunsaturated fatty acid is an omega-3 fatty acid.

11. The method of claim 10, wherein the omega-3 fatty acid is one or more of alpha-linolenic acid, eicosapentaenoic acid, and docosahexaenoic acid.

12. The method of claim 1 wherein the separating of at least a portion of the O₂ from the tail gas is accomplished using one or more stages of pressure swing adsorption, membrane separation, scrubbing with a basic solution, absorption using a solvent, or any combination thereof.

13. The method of claim 1 further comprising recycling the O₂ from the separation of at least a portion of the O₂ from the tail gas to the second bioreactor.

14. The method of claim 1 further comprising recovering a gaseous stream comprising CO₂ and H₂ from the first bioreactor and recycling the gaseous stream to the first bioreactor.

15. The method of claim 1 further comprising recycling at least a portion of the second fermentation broth to the first bioreactor.

16. The method of claim 1 further comprising removing the first microorganism from the first fermentation broth before passing at least a portion of the first fermentation broth to the second bioreactor and recycling the first microorganism to the first bioreactor.

17. The method of claim 1 further comprising removing the second microorganism from the second fermentation broth and recycling the second microorganism to the second bioreactor.

18. The method of claim 17 further comprising passing, to the first bioreactor, the remainder of the second fermentation broth after removing the second microorganism.

19. The method of claim 1 further comprising generating the H₂ using an electrolyzer.

20. The method of claim 1 further comprising generating O₂ using an electrolyzer and introducing the electrolyzer-generated O₂ to the second bioreactor.

21. The method of claim 1 wherein the second bioreactor is operated in an oxygen limited mode.
